(19) United States
(12) Patent Application Publication
(10) Pub. No.: US 2008/0171394 A1
(43) Pub. Date: Jul. 17, 2008

(54) Method For Diagnosing Multiple Sclerosis

(75) Inventors: Bodil Eriksson, Sodertalje (SE); Bo Franzen, Sodertalje (SE); Jan Ottervald, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/995,039

(22) PCT Filed: Jul. 10, 2006
(86) PCT No.: PCT/SE2006/000866

§ 371 Date: Jan. 8, 2008

(30) Foreign Application Priority Data
Jul. 11, 2005 (SE) 0501643-1

(51) Int. Cl. G01N 33/00

(52) U.S. Cl. 436/63; 436/86

(57) ABSTRACT

The present invention relates to methods for diagnosing multiple sclerosis in a subject, the method, comprising determining the level of phosphorylation of a marker in a biological sample from the subject, wherein the marker is selected from α1-antitrypsin (a1AT) and vitamin D binding protein (VDBP); and comparing the level of phosphorylation of the marker in the sample to a reference value.

### FIELD OF THE INVENTION

The present invention relates to biological markers for Multiple Sclerosis. More specifically, the present invention relates to the use of such markers to diagnose Multiple Sclerosis, to monitor progression of the disease and in a clinical or preclinical trial, as well as for drug screening and drug development.

### BACKGROUND OF THE INVENTION

Multiple Sclerosis (MS) is an autoimmune disease involving the nervous system and the disease affects twice as many women as it does men worldwide nearly 2.5 million individuals. In the western world, more than 80 per 100,000 populations are affected. The mean age of onset for MS is 30 years; there are two prevalent age groups. The majority of the patients are between 21 and 25 years at onset and a smaller percentage are 41 to 45 years of age. [Krutze, J F., Neurology 30, 60-79 (1980)]. There is a high economic burden associated with the disease. The total annual cost for all people with MS in the US has been estimated to be more than $9 billion dollars. [Whetten-Goldstein et al., Mult Scler 4, 419-425 (1998)].

MS can be divided into four different forms; clinical isolated syndrome (CIS), relapsing remitting (RR), secondary progressive (SP) and primary progressive (PP) respectively. CIS can be the first step in developing the disease from which 30-80% actually develops MS. [Reviewed by David Miller et al., Clinically isolated syndromes suggestive of multiple sclerosis, part I: natural history, pathogenesis, diagnosis, and prognosis, Neurology, 4, 5, 281-288 (2005)]. RR is characterized by a series of exacerbations that result in varying degrees of disability from which the patient recovers. The course of the disease in about 60-80% of RR patients steadily changes to SP in which the patient does not experience exacerbations, but instead reports a gradual decline. PP does not include the typical exacerbations as in RR instead the disease progression gradually progress.

MS is a chronic demyelinating disease in which inflammation of the CNS is associated with lesions appearing typically in plaques within white matter. This inflammatory process involves activation and recruitment of T cells, macrophages and microglia to lesion sites. Symptoms are believed to occur from axonal demyelination that inhibits or blocks conduction throughout the nervous system. Plaques may be found throughout the brain and spinal cord. Recovery of symptoms has been attributed to partial remyelination and resolution of inflammation. Based on accumulating data from immunological studies of MS patients and a wealth of animal model data, autoimmune dysregulation has been viewed as the major contributor to tissue damage.

The current model of MS immunopathology suggests that autoreactive T cells within the periphery become activated. [Noseworthy, J. et al., N Engl J Med 343, 938-952 (2000)]. Activated T cells express up-regulated levels of adhesion molecules and are able to migrate across the blood-brain barrier much more efficiently than naive, resting T cells. Extravasation across the blood-brain barrier is thought to involve a sequence of overlapping molecular interactions between inducible ligand-receptor pairs on the surface of the migrating cell and the endothelial barrier. Selective expression of adhesion molecules, chemokines and chemokine receptors and matrix metalloproteinases are likely to be important in mediating the transmigration of effector cells across the blood-brain barrier and into the central nervous system (CNS) perivascular tissue in demyelinating diseases.

The pathogenic mechanisms of MS may not be limited to autoimmunity. [Hemmer, B. et al., Nat Rev Neurosci 3, 291-301 (2002)]. Demyelination may occur through many proposed mechanisms: Fas/Fas ligand interactions, toxic cytokines, reactive oxygen species, antibody dependent cellular toxicity and metabolic instability of oligodendrocytes. In addition, axonal damage is increasingly recognized as a prominent pathological feature in MS lesions as well as in normal appearing white matter in MS brains. Whereas these observations do not preclude the role of inflammatory demyelination in MS pathogenesis, axonal compromise may predate the inflammatory lesions, raising the possibility that an independent axonal pathology may contribute to the primary pathobiology of the disease. Studies of the mechanisms of axonal damage and neurodegeneration in MS are in their infancy. However, axonal damage may determine clinical outcome to a large extent. CNS tissue destruction markers would be useful not only for inflammatory demyelination but for neurodegenerative processes in MS.

MS is a systemic disease in terms of its autoimmune pathogenesis and a compartmental disease in as much as the end-organ damage is in the CNS. Thus, biomarkers of the disease would most likely be found in the CSF that surrounds the brain, as well as in other more easily obtainable fluids, such as serum or urine, that are reflective of systemic disease.

The availability of treatments that favourably impact the early course of MS underscores the importance of timely and accurate diagnosis. Currently, the diagnosis of MS is time consuming, expensive and uncertain especially in the early stages of disease. MRI has also been used to assess MS disease activity, disease burden and the dynamic evolution of these parameters over time. [Bourdette, D. et al., J Neuroimmunol 98, 16-21 (1999)]. Serial MRI studies have unequivocally demonstrated that clinically apparent changes reflect only a minor component of disease activity. Overall MRI is limited in its ability to provide specific information about pathology in MS. In the absence of a specific defining assay, the diagnosis of MS continues to be predicated on the clinical history and neurological exam, though use of the MRI has had a major impact on early diagnosis. [McDonald, W. I. et al., Ann Neurol 50, 121-127 (2001)].

Post-translational modifications such as glycosylation patterns may enable the origin of subsets of these proteins to be distinguished. [Hoffmann, A. et al., FEBS Lett 359, 164-168 (1995); Grunewald, S. et al., Biochim Biophys Acta 1455, 54-60 (1999)].

The disease course of MS is highly variable within and between patients indicating that there is disease heterogeneity. Indeed, heterogeneity in MS lesions has been shown in MRI and pathologic studies. MRI affords the ability to identify atrophy and different types of lesions, however it lacks pathologic specificity. Because of its intimate association with the CNS, considerable efforts have been made to identify prognostic and diagnostic markers in the CSF from patients with MS.

Phosphorylation of proteins is also regarded as a post-translational modification that can act as on or off signal for protein action. [Discussed in Principles of interleukin (IL)-6-type cytokine signalling and its regulation by. Heinrich, P, et al. J374, 1-20. (2003)]. The area, phosphorylations and glycosylations, with a proteomics approach on CSF has not been well investigated although some studies shows examples. [Yuko Ogata, M. et al., Journal of Proteome Research, 4, 837-845 837 (2005)]

Characterization of proteins in CSF with proteomic approaches has been sparse. Many of the published studies employ 2-D electrophoresis, which is rather cumbersome and typically requires more protein than routinely can be obtained with CSF. Furthermore, proteins showing extreme low- or high molecular-weight, high hydrophobicity, low abundance and the entire metabolome are not amenable to electrophoresis. [Manabe, T., Electrophoresis 21, 1116-1122 (2000)]. Poor sensitivity has hampered some studies; others have used very large amounts of fluid to compensate. These efforts have yielded identification of a very limited number of proteins. [Puchades, M., et al., Rapid Commun Mass Spectrom 13, 2450-2455 (1999)]. Nonetheless, employing 2-D electrophoresis proteomics and discovery driven strategies, researchers have identified candidate or potential biomarkers within CSF. For example, a complement factor was identified in the CSF of patients with cerebral arteriopathy. [Unlu, M. et al., Neurosci Lett 282, 149-152 (2000)]. Another example is a decreased protein expression in Parkinson's disease patients. [E. J. Finehout et al. Disease Markers, 21, 2, 93-101 (2005)].

In the context of the present invention, the inventors have used depletion of Albumin and Ig G combined with fluorescent stains for total protein and phospho-proteins. This is a novel approach for quantification of phosphor-proteins in CSF from MS patients.

### SUMMARY OF THE INVENTION

The present invention provides biological markers (“biomarkers”) indicative of Multiple Sclerosis (MS). These biomarkers can be used to diagnose the disease, monitor its progression, assess response to therapy and screen drugs for treating MS. Early diagnosis and knowledge of disease progression could allow early institution of treatment when it is most appropriate and would be of the greatest benefit to the patient. In addition, such information will allow prediction of exacerbations and classification of potential MS subtypes. The ability to evaluate response to therapy will allow the personalized treatment of the disease and provided the basis for clinical trials aimed at evaluating the effectiveness of candidate drugs.

Due to the disease course of MS with a pronounced inflammatory component in the early stage (CIS, RR), followed by significant changes in biology to a neurodegenerative state in the later stages of the disease (SP), it is believed that indicates that these biomarkers can be used in monitoring the development of other neuroinflammatory and/or neurodegenerative disorders. Such neuroinflammatory or neurodegenerative disorders could be, but are not limited to, Parkinson's disease, Alzheimer's Disease, Mild Cognitive Impairment, Dementia, Age-Associated Memory Impairment, Age-Related Cognitive Decline, Disorder(s) associated with neurofibrillar tangle pathologies, Dementia due to Alzheimer's Disease, Dementia due to Schizophrenia, Dementia due to Parkinson's Disease, Dementia due to Creutzfeld-Jacob Disease, Dementia due to Huntington's Disease, Dementia due to Pick's Disease, Stroke, Head Trauma, Spinal Injury, Multiple Sclerosis, Migraine, Pain, Systemic Pain, Localized Pain, Nociceptive Pain, Neuropathic Pain, Urinary Incontinence, Sexual Dysfunction, Premature Ejaculation, Motor Disorder(s), Endocrine Disorder(s), Gastrointestinal Disorder(s), and Vasospasm.

The biomarkers of the present invention include the level of phosphorylation of particular proteins whose measurement values in a biological sample are different (either higher or lower) in a subject with MS as compared to a standard level or reference range established by obtaining measurement values for the biomarker in subjects who do not have the disease (“normal controls”). In preferred embodiments, such difference is statistically significant. Alternatively, CSF from individual patients may be analysed longitudinal, prior to and during treatment. Thereby, a significant chance quantify of given biomarkers reflect response to therapy. In particular, these biomarkers comprise the molecules α-1 antitrypsin (a1AT) and Vitamin D-binding protein (VDBP) and their level of phosphorylation found in CSF.

In one embodiment, the invention provides a method for determining whether a subject has MS. In related embodiments, the invention provides a method for determining whether a subject is more likely than not to have MS, or is more likely to have MS than to have another disease. The method is performed by analysing a biological sample, such as serum or CSF, from the subject; measuring the level of phosphorylation of at least one of the biomarkers in the biological sample; and comparing the measured phosphorylation level with a standard level or reference range. Typically, the standard level or reference range is obtained by measuring the same marker or markers in a normal control or, more preferably, a set of normal controls. Depending upon the difference between the measured level and the standard level or reference range, the patient can be diagnosed as having MS, or as not having MS. As will be appreciated by one of skill in the art, a standard level or reference range is specific to the biological sample at issue. Thus, a standard level or reference range for the marker in serum that is indicative of MS would be expected to be different from the standard level or reference range (if one exists) for that same marker in CSF, urine or another tissue, fluid or compartment. Thus, references herein to measuring biomarkers will be understood to refer to measuring the level of phosphorylation of the biomarker. Furthermore, references herein to comparisons between a marker phosphorylation measurement level and a standard level or reference range will be understood to refer to such levels or ranges for the same type of biological sample.

In another embodiment, the invention provides a method for monitoring a MS patient over time to determine whether the disease is progressing. The method is performed by analysing a biological sample, such as serum or CSF, from the subject at a certain time; measuring the phosphorylation level of at least one of the biomarkers in the biological sample; and comparing the measured phosphorylation level with the phosphorylation level measured with respect to a biological sample obtained from the subject at an earlier time. Depending upon the difference between the measured phosphorylation levels, it can be seen whether the marker phosphorylation level has increased, decreased, or remained constant over the interval. Subsequent sample acquisitions and measurements can be performed as many times as desired over a range of times. The same type of method also can be used to assess the efficacy of a therapeutic intervention in a subject where the therapy is instituted, or an ongoing therapy is changed.

In another embodiment, the invention provides a method for conducting a clinical trial to determine whether a candidate drug is effective in treating MS. The method is performed by analysing a biological sample from each subject in a population of subjects diagnosed with MS, and measuring the phosphorylation level of at least one of the biomarkers in the biological samples. Then, a dose of a candidate drug is administered to one portion or sub-population of the same subject population (“experimental group”) while a placebo is administered to the other members of the subject population (“control group”). After drug or placebo administration, a biological sample is acquired from the experimental and control groups and the same assays are performed on the biological samples as were previously performed to obtain phosphorylation measurement values. Depending upon the difference between the measured phosphorylation levels between the experimental and control groups, it can be seen whether the candidate drug is effective. The relative efficacy of two different drugs or other therapies for treating MS can be evaluated using this method by administering the drug or other therapy in place of the placebo. As will be apparent to one of skill in the art, the methods of the present invention may be used to evaluate an existing drug, being used to treat another indication, for its efficacy in treating MS (e.g., by comparing the efficacy of the drug relative to one currently used for treating MS in a clinical trial, as described above).

The present invention also provides molecules that specifically bind to protein and low molecular weight markers. Such marker specific reagents have utility in isolating the markers and in detecting the presence of the markers, e.g., in immunoassays.

The present invention also provides kits for diagnosing MS, monitoring progression of the disease and assessing response to therapy, the kits comprising a container for a sample collected from a subject and at least one marker specific reagent.

### DETAILED DESCRIPTION OF THE INVENTION

The present inventors have surprisingly identified that the level of phosphorylation of certain biological markers whose presence and measurement phosphorylation levels are indicative of multiple sclerosis (MS). The biomarkers include protein and low molecular weight molecules. According to one definition, a biological marker (“biomarker”) is “a characteristic that is objectively measured and evaluated as an indicator of normal biologic processes, pathogenic processes, or pharmacological responses to therapeutic interventions.” NIH Biomarker Definitions Working Group (1998).

Biomarkers can also include patterns or ensembles of characteristics indicative of particular biological processes. The biomarker measurement can increase or decrease to indicate a particular biological event or process. In addition, if a biomarker measurement typically changes in the absence of a particular biological process, a constant measurement can indicate occurrence of that process. For more information on biomarker measurement and discovery, see U.S. patent application Ser. No. 091558, 909, “Phenotype and Biological Marker Identification System,” filed Apr. 26, 2000, herein incorporated by reference in its entirety.

In the present invention, the biomarkers are primarily used for diagnostic purposes. However they may also be used for therapeutic, drug screening and patient stratification purposes (e.g., to group patients into a number of “subsets” for evaluation).

The present invention is based on the findings of a study designed to identify biological markers for MS. Samples of CSF and serum from patients with MS were analyzed using liquid chromatography-mass spectrometry and gas chromatography-mass spectrometry, and the resulting mass spectra profiles were compared. The markers of the present invention were identified by comparing the levels of phosphorylation of markers measured in samples obtained from MS patients with the levels of phosphorylation of markers measured in samples obtained from patients who did not have the disease. Peaks consistently higher or lower in patients with MS were further investigated by using liquid chromatography mass spectrometry (or gas chromatography mass spectrometry) combined with tandem mass spectrometry techniques to identify the molecules at issue.

Measurement of phosphorylation values of the biomarkers was found to differ in biological samples from patients with MS as compared to biological samples from normal controls. In preferred embodiments, such difference was statistically significant. Accordingly, it is believed that these phosphorylated biomarkers are indicators of MS.

The present invention includes all methods relying on correlations between the biomarkers described herein and the presence of MS. In a preferred embodiment, the invention provides methods for determining whether a candidate drug is effective at treating MS by evaluating the effect it has on the biomarker values. In this context, the term “effective” is to be understood broadly to include reducing or alleviating the signs or symptoms of MS, improving the clinical course of the disease, decreasing the number or severity of exacerbations, reducing the number of plaques, reducing the amount or rate of axonal demyelination, reducing the number of inflammatory cells in existing plaque or reducing in any other objective or subjective indicia of the disease. Different drugs, doses and delivery routes can be evaluated by performing the method using different drug administration conditions. The method may also be used to compare the efficacy of two different drugs or other treatments or therapies for MS.

Phosphorylation levels (of the biomarkers) are to be understood as a measurement given from any stain or dye that recognises phosphor groups associated with proteins or peptides, for example Pro-Q Diamond or phosphor specific antibodies (that are used for detection of a1AT and VDBP). The phosphorylation levels can also be measured after purification with different affinity columns such as IMAC or any other phosphor-binding surfaces.

It is expected that the levels of phosphorylation of the biomarkers described herein will be measured in combination with other signs, symptoms and clinical tests of MS, such as MRI scans or MS biomarkers reported in the literature. Likewise, more than one of the biomarkers of the present invention may be measured in combination. Measurement of the phosphorylation of the biomarkers of the invention along with any other markers known in the art, including those not specifically listed herein, falls within the scope of the present invention.

In one embodiment, the present invention provides a method for determining whether a subject has MS. Biomarker phosphorylation level measurements are taken of a biological sample from a patient suspected of having the disease and compared with a standard level or reference range. Typically, the standard biomarker phosphorylation level or reference range is obtained by measuring the same marker or markers in a set of normal controls. Measurement of the standard biomarker phosphorylation level or reference range need not be made contemporaneously; it may be a historical measurement. Preferably the normal control is matched to the patient with respect to some attribute(s) (e.g., age or sex). Depending upon the difference between the measured and standard level or reference range, the patient can be diagnosed as having MS or as not having MS.

What is presently referred to as MS may turn out to be a number of related, but distinguishable conditions. Indeed, four types of MS have already been recognized: (i) benign MS, (ii) relapsing remitting MS, (iii) secondary chronic progressive MS, and (iv) primary progressive MS. Additional classifications may be made, and these types may be further distinguished into subtypes. Any and all of the various forms of MS are intended to be within the scope of the present invention. Indeed, by providing a method for subsetting patients based on biomarker phosphorylation measurement level, the compositions and methods of the present invention may be used to uncover and define various forms of the disease.

The methods of the present invention may be used to make the diagnosis of MS, independently from other information such as the patient's symptoms or the results of other clinical or paraclinical tests. However, the methods of the present invention are preferably used in conjunction with such other data points.

Because a diagnosis is rarely based exclusively on the results of a single test, the method may be used to determine whether a subject is more likely than not to have MS, or is more likely to have MS than to have another disease, based on the difference between the measured and standard level or reference range of the biomarker. Thus, for example, a patient with a putative diagnosis of MS may be diagnosed as being “more likely” or “less likely” to have MS in light of the information provided by a method of the present invention.

The biological sample may be of any tissue or fluid. Preferably, the sample is a CSF or serum sample, but other biological fluids or tissue may be used. Possible biological fluids include, but are not limited to, plasma, urine and neural tissue. CSF represents a preferred biological sample to analyze for MS markers as it bathes the brain and removes metabolites and molecular debris from its liquid environment. Thus, biomolecules associated with the presence and/or progression of MS is expected to be present at highest concentrations in this body fluid. A CSF biomarker in itself may be particularly useful for early diagnosis of disease. Furthermore, molecules initially identified in CSF may also be present, presumably at lower concentrations, in more easily obtainable fluids such as serum and urine. Such biomarkers may be valuable for monitoring all stages of the disease and response to therapy. Serum and urine also represent preferred biological samples as they are expected to be reflective of the systemic manifestations of the disease. In some embodiments, the level of a marker may be compared to the level of another marker or some other component in a different tissue, fluid or biological “compartment.” Thus, a differential comparison may be made of a marker in CSF and serum. It is also within the scope of the invention to compare the level of a marker with the level of another marker or some other component within the same compartment.

As will be apparent to those of ordinary skill in the art, the above description is not limited to making an initial diagnosis of MS, but also is applicable to confirming a provisional diagnosis of MS or “ruling out” such a diagnosis.

As indicated in Tables 2a and 2b (see example section), the marker measurement phosphorylation level values are higher in samples from MS patients. A significant difference in the appropriate direction in the measured value of one or more of the markers indicates that the patient has (or is more likely to have) MS. If only one biomarker phosphorylation level is measured, then that value must increase to indicate MS. Phosphorylation measurements can be of (i) a biomarker of the present invention, (ii) a biomarker of the present invention and another factor known to be associated with MS (e.g., MRI scan); (iii) a plurality of biomarkers comprising at least one biomarker of the present invention and at least one biomarker reported in the literature, or (iv) any combination of the foregoing. Furthermore, the amount of change in a biomarker level may be an indication of the relatively likelihood of the presence of the disease.

The present invention provides phosphorylated biomarkers that the present inventors have shown to be indicative of MS in a subject.

It is to be understood that any correlations between biological sample measurements of these biomarkers and MS, as used for diagnosis of the disease or evaluating drug effect, are within the scope of the present invention.

In the methods of the invention, phosphorylated biomarker levels are measured using conventional techniques. A wide variety of techniques are available, including mass spectrometry, chromatographic separations, 2-D gel separations, binding assays (e.g., immunoassays), competitive inhibition assays, and so on. Any effective method in the art for measuring the level of a protein or low molecular weight marker is included in the invention. It is within the ability of one of ordinary skill in the art to determine which method would be most appropriate for measuring a specific marker. Thus, for example, a robust ELISA assay may be best suited for use in a physician's office while a measurement requiring more sophisticated instrumentation may be best suited for use in a clinical laboratory. Regardless of the method selected, it is important that the measurements be reproducible.

The phosphorylated markers of the invention can be measured by mass spectrometry, which allows direct measurements of analytes with high sensitivity and reproducibility. A number of mass spectrometric methods are available and could be used to accomplish the measurement. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Moreover, mass spectrometers that allow time-of-flight (TOF) measurements have high accuracy and resolution and are able to measure low abundant species, even in complex matrices like serum or CSF.

For protein markers, quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In certain embodiments, the phosphorylated markers are measured using mass spectrometry in connection with a separation technology, such as liquid chromatography-mass spectrometry or gas chromatography-mass spectrometry. It is preferable to couple reverse-phase liquid chromatography to high resolution, high mass accuracy ESI time-of-flight (TOF) mass spectroscopy. This allows spectral intensity measurement of a large number of biomolecules from a relatively small amount of any complex biological material without sacrificing sensitivity or throughput. Analyzing a sample will allow the marker (specified by a specific retention time and m/z) to be determined and quantified. As will be appreciated by one of skill in the art, many other separation technologies may be used in connection with mass spectrometry. For example, a vast array of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass- to-charge ratio (m/z) and retention time (r.t.). The presence of a peak with the m/z and retention time of a biomarker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. In addition, deconvoluting software is available to separate overlapping peaks. The retention time depends to some degree on the conditions employed in performing the liquid chromatography separation.

The better the mass assignment, the more accurate will be the detection and measurement of the marker level in the sample. Thus, the mass spectrometer selected for this purpose preferably provides high mass accuracy and high mass resolution. The mass accuracy of a well-calibrated Micromass TOF instrument, for example, is reported to be approximately 2 mDa, with resolution m/Am exceeding 5000.

In other embodiments, the level of phosphorylation of the markers may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker such as Affibody polypeptides). Standard protocols and data analysis are used to determine the marker concentrations from the assay data.

A number of the assays discussed above employ a reagent that specifically binds to the phosphorylated marker (“marker specific reagent”). Any molecule that is capable of specifically binding to a marker is included within the invention. In some embodiments, the marker specific reagents are antibodies or antibody fragments. In other embodiments, the marker specific reagents are non-antibody species. Thus, for example, a marker specific reagent may be an enzyme for which the marker is a substrate. The marker specific reagents may recognize any epitope of the targeted markers.

A marker specific reagent may be identified and produced by any method accepted in the art. Methods for identifying and producing antibodies and antibody fragments specific for an analyte are well known. Examples of other methods used to identify marker specific reagents include binding assays with random peptide libraries (e.g., phage display) and design methods based on an analysis of the structure of the marker.

The markers of the invention, especially the low molecular weight markers, also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

In an alternative embodiment of the invention, a method is provided for monitoring an MS patient over time to determine whether the disease is progressing. The specific techniques used in implementing this embodiment are similar to those used in the embodiments described above. The method is performed by obtaining a biological sample, such as serum or CSF, from the subject at a certain time (t 1); measuring the level of phosphorylation of at least one of the biomarkers in the biological sample; and comparing the measured level with the phosphorylation level measured with respect to a biological sample obtained from the subject at an earlier time. Depending upon the difference between the measured levels, it can be seen whether the marker phosphorylation level has increased, decreased, or remained constant over the interval. A further deviation of a marker in the direction indicating MS, or the measurement of additional increased or decreased MS markers, would suggest a progression of the disease during the interval. Subsequent sample acquisitions and measurements can be performed as many times as desired over a range of times.

The ability to monitor a patient by making serial marker level phosphorylation determinations would represent a valuable clinical tool. Rather than the limited “snapshot” provided by a single test, such monitoring would reveal trends in marker phosphorylation levels over time. In addition to indicating a progression of the disease, tracking the marker phosphorylation levels in a patient could be used to predict exacerbations or indicate the clinical course of the disease. For example, as will be apparent to one of skill in the art, the biomarkers of the present invention could be further investigated to distinguish between any or all of the known forms of MS (benign MS, relapsing remitting MS, secondary chronic progressive MS, and primary progressive MS) or any later described types or subtypes of the disease. In addition, the sensitivity and specificity of any method of the present invention could be further investigated with respect to distinguishing MS from other diseases of autoimmunity, or other nervous system disorders, or to predict relapse and remission.

Analogously, the phosphorylated markers of the present invention can be used to assess the efficacy of a therapeutic intervention in a subject. The same approach described above would be used, except a suitable treatment would be started, or an ongoing treatment would be changed, before the second measurement. The treatment can be any therapeutic intervention, such as drug administration, dietary restriction or surgery, and can follow any suitable schedule over any time period. The measurements before and after could then be compared to determine whether or not the treatment had an effect effective. As will be appreciated by one of skilled in the art, the determination may be confounded by other superimposed processes (e.g., an exacerbation of the disease during the same period).

In a further additional embodiment, the phosphorylated markers may be used to screen candidate drugs in a clinical trial to determine whether a candidate drug is effective in treating MS. At time t 0, a biological sample is obtained from each subject in population of subjects diagnosed with MS. Next, assays are performed on each subject's sample to measure phosphorylation levels of a biological marker. In some embodiments, only a single marker is monitored, while in other embodiments, a combination of markers, up to the total number of factors, is monitored. Next, a predetermined dose of a candidate drug is administered to a portion or sub-population of the same subject population. Drug administration can follow any suitable schedule over any time period. In some cases, varying doses are administered to different subjects within the sub-population, or the drug is administered by different routes. After drug administration, a biological sample is acquired from the sub-population and the same assays are performed on the biological samples as were previously performed to obtain phosphorylation measurement values. As before, subsequent sample acquisitions and measurements can be performed as many times as desired over a range of times. In such a study, a different subpopulation of the subject population serves as a control group, to which a placebo is administered. The same procedure is then followed for the control group: obtaining the biological sample, processing the sample, and measuring the phosphorylation of the biological markers to obtain a measurement chart.

Specific doses and delivery routes can also be examined. The method is performed by administering the candidate drug at specified dose or delivery routes to subjects with MS; obtaining biological samples, such as serum or CSF, from the subjects; measuring the phosphorylation level of at least one of the biomarkers in each of the biological samples; and, comparing the measured phosphorylation level for each sample with other samples and/or a standard phosphorylation level. Typically, the standard phosphorylation level is obtained by measuring the same marker or markers in the subject before drug administration. Depending upon the difference between the measured and standard phosphorylation levels, the drug can be considered to have an effect on MS. If multiple biomarkers are measured, at least one and up to all of the biomarkers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating MS.

In a typical embodiment, a subject population having MS is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed. Alternatively, subgroups may be defined by the phosphorylation level of biomarkers.

In general, a number of statistical considerations must be made in designing the trial to ensure that statistically significant changes in biomarker phosphorylation measurements can be detected following drug administration. The amount of change in a biomarker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. A biological sample is obtained from each subject in the population. Preferably, the sample is blood or CSF, but other biological fluids may be used (e.g., urine). Next, an assay or variety of assays is performed on each subject's sample to measure phosphorylation levels of particular biomarkers of the invention. The assays can use conventional methods and reagents, as described above. If the sample is blood, then the assays typically are performed on either serum or plasma. For other fluids, additional sample preparation steps are included as necessary before the assays are performed. The assays measure values of at least one of the biological markers described herein. In some embodiments, only a single marker is monitored, while in other embodiments, a combination of factors, up to the total number of markers, is monitored. The markers may also be monitored in conjunction with other measurements and factors associated with MS (e.g., MRI imaging). The number of biological markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. After drug administration, another biological sample is acquired from the sub-population. Typically, the sample is the same type of sample and processed in the same manner (for example, CSF or blood) as the sample acquired from the subject population before drug administration (the “t0 sample”). The same assays are performed on the samples to obtain measurement values. Subsequent sample acquisitions and measurements can be performed as many times as desired over a range of times.

Typically, a different sub-population of the subject population is used as a control group, to which a placebo is administered. The same procedure is then followed for the control group: obtaining the biological sample, processing the sample, and measuring the phosphorylation of biological markers to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired phosphorylation measurements of the various biomarkers are now available for each subject. The different phosphorylation measurement values are compared and analyzed to determine whether the biological markers changed in the expected direction for the drug group but not for the placebo group, indicating that the candidate drug is effective in treating the disease. In preferred embodiments, such change is statistically significant. The measurement values for the group that received the candidate drug are compared with standard measurement values, preferably the measured values before the drug was given to the group. Typically, the comparison takes the form of statistical analysis of the measured phosphorylation values of the entire population before and after administration of the drug or placebo. Any conventional statistical method can be used to determine whether the changes in phosphorylation of the biological marker values are statistically significant. For example, paired comparisons can be made for each biomarker using either a parametric paired t-test or a non-parametric sign or sign rank test, depending upon the distribution of the data.

In addition, tests should be performed to ensure that statistically significant changes found in the drug group are not also found in the placebo group. Without such tests, it cannot be determined whether the observed changes occur in all patients and are therefore not a result of candidate drug administration.

If phosphorylation of only one biomarker is measured, then that value must increase to indicate drug efficacy. If more than one biomarker is measured, then drug efficacy can be indicated by change in only one biomarker, all biomarkers, or any number in between. In some embodiments, multiple markers are measured, and drug efficacy is indicated by changes in multiple markers. Phosphorylation measurements can be of both biomarkers of the present invention and other measurements and factors associated with MS (e.g., measurement of biomarkers reported in the literature and/or MRI imaging). Furthermore, the amount of change in a biomarker phosphorylation level may be an indication of the relatively efficacy of the drug.

In addition to determining whether a particular drug is effective in treating MS, biomarkers of the invention can also be used to examine dose effects of a candidate drug. There are a number of different ways that varying doses can be examined. For example, different doses of a drug can be administered to different subject populations, and phosphorylation measurements corresponding to each dose analyzed to determine if the differences in the inventive biomarkers before and after drug administration are significant. In this way, a minimal dose required to effect a change can be estimated. In addition, results from different doses can be compared with each other to determine how each biomarker behaves as a function of dose.

Analogously, administration routes of a particular drug can be examined. The drug can be administered differently to different subject populations, and phosphorylation measurements corresponding to each administration route analyzed to determined if the differences in the inventive biomarkers before and after drug administration are significant. Results from the different routes can also be compared with each other directly.

The present invention also provides kits for diagnosing MS, monitoring progression of the disease and assessing response to therapy. The kits comprise a container for sample collected from a patient and a marker specific reagent. In developing such kits, it is within the competence of one of ordinary skill in the art to perform validation studies that would use an optimal analytical platform for each marker. For a given marker, this may be an immunoassay or mass spectrometry assay. Kit development may require specific antibody development, evaluation of the influence (if any) of matrix constituent (“matrix effects”), and assay performance specifications. It may turn out that a combination of two or more markers provides the best specificity and sensitivity, and hence utility for monitoring the disease.

Any of the methods described herein may be used in conjunction with other methods of diagnosing, monitoring and subsetting. The description of the methods herein makes reference to measuring phosphorylation of “a marker.” Typically, however a single marker may not be sufficient to provide a definitive diagnosis of a disease. In a preferred embodiment, the methods of the invention involve measuring phosphorylation of two markers, said markers being a1AT and VDBP.

Accordingly, one aspect of the present invention relates to a method for diagnosing multiple sclerosis in a subject, the method, comprising determining the level of phosphorylation of a marker in a biological sample from the subject, wherein the marker is selected from α1-antitrypsin (a1AT) and vitamin D binding protein (VDBP); and comparing the level of phosphorylation of the marker in the sample to a reference value.

In another aspect of the present invention, said method is being carried out in vitro.

In yet another aspect of the present invention, the biological sample is a body fluid.

In yet another aspect of the present invention, said body fluid is selected from the group consisting of blood, serum, plasma, cerebrospinal fluid, urine, and saliva.

In yet another aspect of the present invention, said marker is a1AT.

In yet another aspect of the present invention, said marker is VDBP.

In yet another aspect of the present invention, said marker is a1AT and VDBP.

In yet another aspect of the present invention, said reference value is the phosphorylation level of the marker in at least one sample from a non-multiple sclerosis subject.

In yet another aspect of the present invention, the level of phosphorylation of the marker is determined by detecting the presence of a metabolite.

In yet another aspect of the present invention, said subject is a lab animal.

In yet another aspect of the present invention, said subject is a human subject.

In yet another aspect of the present invention, there is provided method for monitoring the progression of multiple sclerosis in a subject, the method comprising measuring the level of phosphorylation of a marker in a biological sample from the subject in a first sample, wherein the marker is selected from the group consisting of a1AT and VDBP; measuring the level of phosphorylation of the marker in a biological sample from a second sample; and comparing the phosphorylation level of the marker measured in the first sample with the phosphorylation level of the marker measured in the second sample.

In yet another aspect of the present invention, there is provided method for aiding in the diagnosis of multiple sclerosis in a subject, the method comprising determining the phosphorylation level of a marker in a biological sample from the subject, wherein the marker is selected from the group consisting of a1AT and VDBP; comparing the phosphorylation level of the marker in the sample to a reference value; and determining from the results of the comparison whether the subject is more or less likely to have multiple sclerosis.

In yet another aspect of the present invention, there is provided a method of assessing the efficacy of a treatment for multiple sclerosis in a subject, the method comprising comparing: (i) the phosphorylation level of a marker measured in a first sample obtained from the subject before the treatment has been administered to the subject, wherein the marker is selected a1AT and VDBP; and (ii) the phosphorylation level of the marker in a second sample obtained from the subject after the treatment has been administered to the subject, wherein a decrease in the phosphorylation level of the marker in the second sample relative to the first sample is an indication that the treatment is efficacious for treating multiple sclerosis in the subject.

In yet another aspect of the present invention, there is provided a method for determining the type, stage or severity of multiple sclerosis in a subject, the method comprising determining the phosphorylation level of a marker in a biological sample from the subject, wherein the marker is selected from the group consisting of a1AT and VDBP; comparing the phosphorylation level of the marker in the sample to a reference value; and determining from the results of the comparison the type, stage or severity of multiple sclerosis in the subject.

In yet another aspect of the present invention, there is provided a method for determining the risk of developing multiple sclerosis in a subject, the method comprising determining the phosphorylation level of a marker in a biological sample from the subject, wherein the marker is selected from a1AT and VDBP; comparing the phosphorylation level of the marker in the sample to a reference value; and determining from the results of the comparison that the subject has an increased or decreased risk of developing multiple sclerosis.

The invention will now be illustrated by the following non-limiting example:

### EXAMPLE

### Material

Samples were collected at Karolinska Hospital Stockholm, Sweden (provided by Professor Tomas Olsson, CMM, Karolinska Institute, Sweden), during investigation of patients with possible Multiple Sclerosis, diagnosis criteria described in [Recommended Diagnostic Criteria for Multiple Sclerosis: Guidelines from the International Panel on the Diagnosis of Multiple Sclerosis, W. Ian McDonald et al., Ann Neurol; 50, 121-127 (2001)].

CSF samples were acquired with lumbar punction and thereafter the tubes were centrifuged and CSF without cells was frozen until further analysis. Patient information is provided in Table 1.

### Sample Preparation

All CSF samples were affinity purified with POROS anti-HSA column, 2 ml (Applied Biosystems, USA) and a HiTrap Protein G column, 1 ml (GE healthcare, USA) to remove Albumin and IgG respectively. The columns were connected in series during the purification process. The purification was performed on FPLC, LCC-501 PLUS (GE healthcare, USA). The flow rate was 1 ml/min during the procedure. The samples were purified according to manufacturer's instructions. Briefly, the sample was diluted in 10 mM Tris-HCl pH 7.0 (Bio-Rad, Hercules, Calif., USA) binding buffer including Complete™ protease inhibitor cocktail (Roche, Germany) 1:1. Thereafter, the sample was filtered with a 0.45 μm filter (Pall, USA). The solution was thereafter loaded onto the columns with an auto injector. The flow through was collected in 2 ml fractions pooled and frozen until further preparation and analysis. Elution of bound proteins was made with 10 column volumes (CV) 10 mM Tris-HCl, pH 2.0 elution buffer and the column were cleaned with 10 CV 1.0 M NaCl (Merck, USA) of regeneration buffer and finally 10 CV of binding buffer as preparation for a new sample.

### Desalting and Concentration

Individual samples were placed in a 15 ml Amnicon-Ultra spin column with a cut-off at 5 KDa (Millipore, USA). The centrifugation was 3×40′+1×30′ and the sample was diluted in Tris-HCl pH 7 between each centrifugation step. The decrease in salt concentration was estimated to be <2000 times and it was performed in Megafuge2.0 R for 4000 rpm in 4° C. After centrifugation the remaining sample was transferred to 1.5 ml eppendorf tubes (Eppendorf, USA) and speedvaced until dryness. Thereafter the samples were frozen until the following day.

### SDS-PAGE for Detection of Protein Concentration

To adjust for different protein concentrations in the samples, one-dimensional gel electrophoresis followed by image analysis and the total protein concentration were determined. The sample was resolved in 25 μl sample buffer, Nupage LDS Sample Buffer (4× concentration, Invitrogen, USA) with 5% glycerol (BDH laboratory, UK) and 50 mM DTT (DTT, Servan, Germany) added and boiled for 3×15 in 95° C. on a plate heater. Between the boiling steps, samples were mixed accurate for 5′ and after the last boiling step 75 μl Rh (see below) buffer was added to a final volume of 100 μl and the sample was finally mixed for 15′. 1.2 μl of boiled sample was transferred into a 1.5 ml eppendorf tube and 10.8 μl Nupage LDS Sample Buffer (Invitrogen, USA) was added to an final volume of 12 μl. Ten μl were added onto each lane on Novex gradient gels (bis-tris 10 lanes, 4-14%, Invitrogen, USA) The gels were run for 30′ in MES buffer (Invitrogen, USA) at 200V, constant run. Thereafter the gels were put into fixation solution (FS) (20% Ethanol and 7% acetic acid) for more than 30′. To visualise total protein the gels were washed in Milli Q water MQ water and then placed in 75 ml Sypro Ruby® stain (Molecular Probes, Inc. USA) overnight as described by [Kiera Berggren, Elena Chernokalskaya, Thomas H. Steinberg, Courtenay Kemper, Mary F. Lopez, Zhenjum Diwu, Richard P. Haugland, Wayne F. Patton, Proteomics, 1, 54-665, (2001)]. The following day the gels were washed in fixation solution once for 5′ followed by MQ 3×15′, thereafter the gels were scanned with Molecular Imager FX (Bio-Rad, Hercules, Calif., USA) at 100 μm resolution. To adjust for different protein concentrations in the samples a volume report analysis was made with the Quantity One (Bio-Rad, Hercules, Calif., USA).

### Two-Dimensional Gel Electrophoresis

Before isoelectric focusing, samples were wortexed 30′ and centrifuged 15′ at 13000×g to erase insoluble molecules. Thereafter the estimated equal protein amounts in their respectively volume were transferred to 1.5 ml eppendorf tubes and rehydration solution (RH; Urea 8 M (Sigma, USA) DTT 19.5 mM, NP-40 (10%) 0.5% (v/v) (USB Corporation, USA) IPG-buffer 4-7, 0.5% (v/v) (GE healthcare, USA) Glycerol 7% (v/v) CHAPS1.5% (Genomic solutions, USA), thiourea 2M, (Fluka, Germany)) was added to a final volume of 4601 μl. 2 μl BFB was added to give colour. The sample was placed in a porslin tray and the strip was placed with the gel facing down. Thereafter the strip was covered with Plus one mineral oil (GE Healthcare, USA) and a plastic lid was placed on the top. Isoelectric focusing was performed in IPGphor (GE Healthcare, USA) with IPG strips pH4-7 Liner, 24 cm (GE Healthcare, USA) for a total 120 kVhrs. After the run the strips were sealed in plastic bags and stored in −35° C. Prior to the 2-D run, the IPG strips were subjected to a two-step reduction and alkylation step by equilibrating the strips for 15 min first in 50 mM Tris-HCl, pH 6.8, 6 M urea, 30% v/v glycerol, 2% w/v SDS (Bio-Rad, Hercules, Calif., USA), and 65 mM DTT, and then for 15 min in 50 mM Tris-HCl, pH 8.8, 6 M urea, 30% v/v glycerol, 2% w/v SDS, and 259 mM iodoacetamide (IAA, Matrix Scientific, USA). Thereafter the strips were placed on the top of the gel and sealed with heated agarose (1% w/v, Low melting agarose, USB Corporation, USA). The gels were allow to cool down before placed in a Hoefer Isodalt tank with Tris/glyc/SDS running buffer (Bio-Rad, Hercules, Calif., USA) for >19 h on 100 v constant. The 2-D separation was conducted in 1.0×220×200 mm thick 12.5% polyacrylamide gel.

### Protein Staining

For visualisation of phosphorylated proteins the gels were stained with Pro-Q® Diamond (Molecular Probes, Inc. USA) as described by [Birte Schulenberg, Terrie N. Goodman, Robert Aggeler, Roderick A. Capaldi, Wayne F Patton Characterization of dynamic and steady-state protein phosphorylation using a fluorescent phosphoprotein gel stain and mass spectrometry. Electrophoresis, 25(15): 2526-32 (2004)], with some modifications. Briefly, the gels were fixed in FS for 3×30′ followed by rinsing I MQ water 30′ thereafter the gels are placed in plastic containers with Pro-Q® over night protected from light. The gels were washed in destain solution; 20% acetonitril 50 mM sodium acetate 3×30′ followed rinsing in MQ water twice for 5′. Subsequently the gels were scanned with Molecular Imager® FX (Bio-Rad, Hercules, Calif., USA) at 100 μm resolution. Gels were stained using automated strainers (GE Healthcare, USA) for total protein staining with Sypro Ruby®. The autostainer program included the following step; 2× 15′MQ, 3× 30′ fixation, 12 h Sypro Ruby®, 3× 15′MQ for and final step in fixation solution. Thereafter the gels were scanned with Molecular Imager® FX at 100 μm resolution. After scanning the gels is stored in plastic bags in a 0.01% sodium azid solution.

### Image Analysis

Image files (16 bit grey levels and 100 μm resolution) representing SyproRuby® and pro-Q Diamonds® stained gels were processed for background subtraction and protein spot detection using one defined set of parameters. These parameters were optimized using tools given by the software PDQuest (version 7.3, Bio-Rad, Hercules, Calif., USA). Detected protein spots were then matched between gels and a synthetic master image was prepared to represent most of the protein spots present in all gels. The quantity of each protein spot was expressed as ppm (parts per million) of the total sum of the integrated spot volumes of the given gel image. This procedure allow for quantitative comparison of all protein spots detected in all gels. Protein spots of interest were excised from gels using a spot cutter robot (Bio-Rad, Hercules, Calif., USA), transferred to 96-well plates.

### Mass Spectrometry and Protein Identification

A MassPREP robot (Waters Corporation, USA) was used to extract proteins from gel spots by digesting proteins to give peptides for running on a MALDI time-of-flight (MALDI-ToF, Waters Corporation, USA) mass spectrometer. Sypro Ruby®-stained gel pieces were dehydrated using acetonitrile and trypsinated (Trypsin, Promega, USA), followed by 1% formic acid/2% acetonitrile peptide extraction. After peptides were moved to their corresponding positions on another microtiter plate, ZipTips containing C18-gel were used for peptide concentration, desalting and elution together with the spot matrix, (α-cyano-4-hydroxycinnmic acid, Waters Corporation, USA) onto a MALDI target plate, and crystals of matrix and peptide were formed. After MALDI-ToF was used to attain the mass of each peptide and the resulting peaklist was imported into the search engineens Protein Lynx Global Server 2 (PLGS2) and MASCOT, several databases were selected as information sources. Protein modifications were oxidation and carbamidomethyl, and the upper limit for miscleaveage was set to 1 and the peptide charge to +1. The charge on peptides as a result of the MALDI-ToF process was always +1 and peptide tolerance was +/−100 ppm. A more detailed description of these procedures is given in [Dihydropyrimidinase related protein-2 as a biomarker for temperature and time dependent post mortem changes in the mouse brain proteome PROTEOMICS, 3, 10, 1920-1929 (2003) Bo Franzén et al.].

### Results

Nine sample pools were analyzed. Each pool consisted of 2-3 selected patients with diagnosis given according to table 1. The number of patients per sample pool was chosen to give 0.6 mg total protein content of each pool for 2D gel analysis and the patient distribution within each pool was equal, with respect to protein quantity.

Quantitative analysis of disease groups versus controls showed pronounced differences in pro-Q Diamond® staining in two groups of protein spots. Protein spots within these groups were identified by MALDI-TOF MS as alpha-1-antitrypsin (a1AT) and vitamin D binding protein (ViD PB), see Table 3 for details. Six protein spots in the first group of spots were all identified as a1AT and our calculations (Tab 2) were based on their total volume (6A1AT). More than one protein spot was identified as ViD PB, however, only one was selected for quantitative measurements.

Table 2a and 2b show protein levels of a1AT and ViD BP, respectively. Fold change Q columns show an increase of protein phosphorylation in RR and SP pools for both proteins. However, the total protein load on each gel vary as well as the individual expression levels of a1AT and ViD BP. Therefore, we have stained for total protein using SyproRuby® and express the phosphorylation in percent of total stain of each protein analyzed (% Q/S).

The phosphorylation of a1AT was significantly increased in the RR group (p<0.01) and SP group (p<0.05), compared to the OND group. A clear trend of increased phosphorylation was observed in the SP group compared to the RR group, although it was not statistically significant. If this trend is true, the phosphorylation of a1AT may serve as a disease progression marker.

One patient (00-013) from pool SP114 was reclassified after protein analysis as OND (table 1). Table 2a and 2b show that pool SP 114 express % Q/S values below the average of the SP group. Nevertheless, the SP groups (a1AT and ViD BP) showed significant increase compared to the OND groups.

TABLE 1
Samples and clinical information

| | Sample | | | | | | | | | |
|---|---|---|---|---|---|---|---|---|---|---|
| Sample | ID | | | | Disease | | | Onset5 | CSF-IgG | Ig-band and |
| Pool | (patient) | Sex | Age | Diagnos¹ | activity2 | EDSS3 | MRT4 | (year) | index6 | comments7 |
| OND118 | 01-116 | F | 63 | OND | Remission | 2.5 | >5 < 8 | 1965 | 0.58 | Normal oligoklonal Ig |
| | | | | | OND INF | | | | | and normal IgG-index |
| | 02-009 | F | 62 | OND | | | >5 < 8 | 1997 | 0.54 | Normal oligoklonal Ig |
| | | | | | | | | | | and normal IgG-index |
| OND219 | 01-061 | F | 56 | OND/TH | OND INF | | | | 0.52 | Normal oligoklonal Ig |
| | | | | | | | | | | and normal IgG-index |
| | 01-078 | M | 43 | OND | OND INF | | | | 0.76 | Susspect oligoklonal Ig |
| | | | | | | | | | | and increased IgG- |
| | | | | | | | | | | index |
| | 01-164 | F | 47 | OND/TH | OND INF | | | | 0.45 | Normal oligoklonal Ig |
| | | | | | | | | | | and normal IgG-index/ |
| | | | | | | | | | | Barr. Dam. |
| OND320 | 01-077 | M | 38 | OND | OND INF | | | | 0.43 | Normal oligoklonal Ig |
| | | | | | | | | | | and normal IgG-index |
| | 01-185 | M | 51 | OND/TH | | | | | 0.56 | Normal oligoklonal Ig |
| | | | | | | | | | | and normal IgG-index |
| | 02-041 | F | 51 | OND | | 1 | 0 | 2001 | 0.45 | Normal oligoklonal Ig |
| | | | | | | | | | | and normal IgG-index |
| RR111 | 02-021 | M | 21 | CDMS/RR | Remission | 2 | >9 | 2001 | 0.44 | Oligoklonal Ig but |
| | | | | | | | | | | normal IgG-index |
| | 02-037 | F | 47 | CDMS/RR | Relapse | 2.5 | | | 0.55 | Few oligoklonal IgG- |
| | | | | | | | | | | band but normal IgG- |
| | | | | | | | | | | index/Barr. dam. |
| | 02-038 | F | 35 | CDMS/RR | | 0 | >9 | 1999 | 1.11 | Oligoklonal Ig and |
| | | | | | | | | | | inceased IgG-index |
| RR212 | 01-009 | F | 40 | CDMS/RR | | 3 | <2 | 1999 | 1.19 | Oligoklonal Ig and |
| | | | | | | | | | | inceased IgG-index |
| | 02-025 | F | 29 | CDMS/RR | Remission | 1 | >9 | 1996 | 2.51 | Oligoklonal Ig and |
| | | | | | | | | | | inceased IgG-index |
| RR313 | 02-020 | F | 38 | CDMS/RR | Relapse | 2 | <2 | 2002 | 0.84 | Oligoklonal Ig and |
| | | | | | | | | | | inceased IgG-index |
| | 02-023 | F | 46 | CDMS/RR | Remission | 1.5 | >9 | 1974 | 0.99 | Oligoklonal Ig and |
| | | | | | | | | | | inceased IgG-index |
| | 02-046 | F | 38 | CDMS/RR | | 4.0 | | 1997 | 1.28 | Oligoklonal Ig and |
| | | | | | | | | | | inceased IgG-index |
| SP114 | 00-013 | M | 46 | CDMS/SP | SP->OND | | 0 | | 0.41 | Normal oligoklonal Ig |
| | | | | | | | | | | and normal IgG-index |
| | 01-123 | M | 45 | CDMS/SP | | 6.5 | >9 | 1993 | ND | ND |
| SP215 | 00-015 | F | 45 | CDMS/SP | | 3.5 | 0 | 1987 | 0.52 | Oligoklonal Ig but |
| | | | | | | | | | | normal IgG-index/Barr. |
| | | | | | | | | | | dam. |
| | 00-087 | M | 51 | CDMS/SP | | 4.5 | >5 < 8 | 1989 | 0.51 | Normal oligoklonal Ig |
| | | | | | | | | | | and normal IgG-index |
| | 01-010 | F | 82 | CDMS/SP | | 6 | ND | 1952 | 0.55 | Susspect oligoklonal Ig |
| | | | | | | | | | | but normal IgG-index |
| SP316 | 00-110 | F | 64 | CDMS/SP | | 5.0 | 0 | 1979 | 0.46 | Normal oligoklonal Ig |
| | | | | | | | | | | and normal IgG-index |
| | 01-107 | F | 53 | CDMS/SP | | 3.5 | >5 < 8 | 1973 | 0.6 | Oligoklonal Ig but |
| | | | | | | | | | | normal IgG-index |
| | 01-131 | F | 39 | CDMS/SP | | 4.5 | >9 | 1978 | ND | ND |

¹OND = other neurological disease, TH = tension headache, CDMS = clinically definite multiple sclerosis, RR = relapsing remitting, SP = secondary progressive
2INF = inflammation
3Expanded Disability Status Score
4Number of magnet resonance imaging detected lesions, ND = not determined
5Year of clinical onset, first clinical signs of disease.
6IgG index defines as: ([IgG]CSF/[IgG]plasma)/([albumin] CSF/[albumin] plasma), if >0.7, then barrier damage ND = not determined
7Barr.dam. = blood brain barrier damage, ND = not determined

TABLE 2A
Levels of A1AT

| | | | Fold | | | | |
|---|---|---|---|---|---|---|---|
| Sample | | | change | | | | Fold change |
| pool1) | Sum6A1AT_Q2) | Average3) | Q4) | Sum6A1AT_S5) | % Q/S6) | Average7) | % Q/S8) |
| OND118 | 7500 | | | 89732 | 8.36 | | |
| OND219 | 13543 | 9412.36 | 1.00 | 88179 | 15.36 | 11.06 | 1.00 |
| OND320 | 7194 | | | 76097 | 9.45 | | |
| RR111 | 34947 | | | 100956 | 34.62 | | |
| RR212 | 38162 | 31163.54 | 3.31 | 144827 | 26.35 | 29.02**) | 2.62 |
| RR313 | 20381 | | | 78138 | 26.08 | | |
| SP114 | 51775 | | | 99899 | 51.83 | | |
| SP215 | 22289 | 39102.99 | 4.15 | 58827 | 37.89 | 60.14*) | 5.44 |
| SP316 | 43244 | | | 47675 | 90.71 | | |

1)Sample equals a pool of 2 or 3 patient samples. Each patient is described in TABLE 1
2)The sum of 6 integrated Pro-Q Diamond ® stained ppm normalized spot volumes corresponding spots SSP1517, SSP2502, SSP2511, SSP2512, SSP2513 and SSP3520
3)The average of tree values from column “Sum6A1AT_Q”
4)Fold change equals the quote between average values for Pro-Q Diamond ® staining
5)The sum of 6 integrated Sypro Ruby ® stained ppm normalized spot volumes corresponding spots SSP1517, SSP2502, SSP2511, SSP2512, SSP2513 and SSP3520
6)The percentage Pro-Q Diamond ® stain [Sum6A1AT_Q] of Sypro Ruby ® (total protein) [Sum6A1AT_S] for each sample pool
7)The average of tree values from column “% Q/S”
8)Fold change equals the quote between average values for “% Q/S”
*)P < 0.05 (Student's t-test)
**)P < 0.01 (Student's t-test)

TABLE 2B
Levels of ViD BP

| | | | Fold | | | | |
|---|---|---|---|---|---|---|---|
| Sample | | | change | | | | Fold change |
| pool1) | SSP3516_Q2) | Average3) | Q4) | SSP3516_S5) | % Q/S6) | Average7) | % Q/S8) |
| OND118 | 277 | | | 8769 | 3.16 | | |
| OND219 | 673 | 438.26 | 1.00 | 6689 | 10.06 | 6.25 | 1.00 |
| OND320 | 364 | | | 6601 | 5.52 | | |
| RR111 | 2403 | | | 5345 | 44.96 | | |
| RR212 | 3324 | 2273.56 | 5.19 | 9103 | 36.52 | 32.55*) | 5.21 |
| RR313 | 1093 | | | 6767 | 16.16 | | |
| SP114 | 1423 | | | 6630 | 21.46 | | |
| SP215 | 1100 | 1921.94 | 4.39 | 4232 | 26.00 | 47.95*) | 7.67 |
| SP316 | 3243 | | | 3364 | 96.41 | | |

1)Sample equals a pool of 2 or 3 patient samples. Each patient is described in TABLE 1
2)The integrated Pro-Q Diamond ® stained ppm normalized spot volume corresponding spot SSP3516
3)The average of tree values from column “SSP3516_Q”
4)Fold change equals the quote between average values for Pro-Q Diamond ® staining
5)The integrated Sypro Ruby ® stained ppm normalized spot volume corresponding spot SSP3516
6)The percentage Pro-Q Diamond ® stain [SSP3516 Q] of Sypro Ruby ® (total protein) stain [SSP3516_S] for each sample pool
7)The average of tree values from column “% Q/S”
8)Fold change equals the quote between average values for “% Q/S”
*)P < 0.05 (Studen's t-test)

TABLE 3

Identification of protein spots from
2D gel using MALDI-TOF mass spectrometry

| 2D-gel spot | 2D-gel spot number | | |
|---|---|---|---|
| number | SSP 2513 | SSP 2502 | SSP 3516 |

| | | | |
|---|---|---|---|
| Protein Name | Alpha-1-antitrypsin | Alpha-1-antitrypsin | Vitamin D-binding |
| | precursor | precursor | protein precursor |
| Swiss PROT | A1AT_HUMAN | A1AT_HUMAN | VTDB_HUMAN |
| Accession | | | |
| PLGS2 Score | 11.996 | 11.996 | 11.995 |
| PLGS2 % | 99.90886 | 99.90886 | 99.80896 |
| Probability | | | |
| PLGS2 Peptides | 19 | 17 | 9 |
| matched | | | |
| PLGS2 | 44.737% | 44.498 | 22.574 |
| Sequence | | | |
| coverage | | | |
| Mascot Score | 179 | 178 | 141 |
| (>65 = significant) | | | |
| Mascot Peptides | 15 | 15 | 9 |
| matched | | | |
| Sequence | 42% | 42% | 22% |
| Coverage: | | | |
| Nominal mass | 46878 | 46878 | 54526 |
| (Mr): | | | |
| Calculated pl | 5.37 | 5.37 | 5.40 |
| value: | | | |
| Matched peptide | MPSSVSWGIL | MPSSVSWGIL | MKRVLVLLLA |
| sequences | LLAGLCCLVP | LLAGLCCLVP | VAFGHALERG |
| - shown in Bold | VSLAEDPQGD | VSLAEDPQGD | RDYEKNKVCK |
| Black | AAQKTDTSHH | AAQKTDTSHH | EFSHLGKEDF |
| | DQDHPTFNKI | DQDHPTFNKI | TSLSLVLYSR |
| | TPNLAEFAFS | TPNLAEFAFS | KFPSGTFEQV |
| | LYRQLAHQSN | LYRQLAHQSN | SQLVKEVVSL |
| | STNIFFSPVS | STNIFFSPVS | TEACCAEGAD |
| | IATAFAMLSL | IATAFAMLSL | PDCYDTRTSA |
| | GTKADTHDEI | GTKADTHDEI | LSAKSCESNS |
| | LEGLNFNLTE | LEGLNFNLTE | PFPVHPGTAE |
| | IPEAQIHEGF | IPEAQIHEGF | CCTKEGLERK |
| | QELLRTLNQP | QELLRTLNQP | LCMAALKHQP |
| | DSQLQLTTGN | DSQLQLTTGN | QEFPTYVEPT |
| | GLFLSEGLKL | GLFLSEGLKL | NDEICEAFRK |
| | VDKFLEDVKK | VDKFLEDVKK | DPKEYANQFM |
| | LYHSEAFTVN | LYHSEAFTVN | WEYSTNYGQA |
| | FGDTEEAKKQ | FGDTEEAKKQ | PLSLLVSYTK |
| | INDYVEKGTQ | INDYVEKGTQ | SYLSMVGSCC |
| | GKIVDLVKEL | GKIVDLVKEL | TSASPTVCFL |
| | DRDTVFALVN | DRDTVFALVN | KERLQLKHLS |
| | YIFFKGKWER | YIFFKGKWER | LLTTLSNRVC |
| | PFEVKDTEEE | PFEVKDTEEE | SQYAAYGEKK |
| | DFHVDQVTTV | DFHVDQVTTV | SRLSNLIKLA |
| | KVPMMKRLGM | KVPMMKRLGM | QKVPTADLED |
| | FNIQHCKKLS | FNIQHCKKLS | VLPLAEDITN |
| | SWVLLMKYLG | SWVLLMKYLG | ILSKCCESAS |
| | NATAIFFLPD | NATAIFFLPD | EDCMAKELPE |
| | EGKLQHLENE | EGKLQHLENE | HTVKLCDNLS |
| | LTHDIITKFL | LTHDIITKFL | TKNSKFEDCC |
| | ENEDRRSASL | ENEDRRSASL | QEKTAMDVFV |
| | HLPKLSITGT | HLPKLSITGT | CTYFMPAAQL |
| | YDLKSVLGQL | YDLKSVLGQL | PELPDVELPT |
| | GITKVFSNGA | GITKVFSNGA | NKDVCDPGNT |
| | DLSGVTEEAP | DLSGVTEEAP | KVMDKYTFEL |
| | LKLSKAVHKA | LKLSKAVHKA | SRRTHLPEVF |
| | VLTIDEKGTE | VLTIDEKGTE | LSKVLEPTLK |
| | AAGAMFLEAI | AAGAMFLEAI | SLGECCDVED |
| | PMSIPPEVKF | PMSIPPEVKF | STTCFNAKGP |
| | NKPFVFLMIE | NKPFVFLMIE | LLKKELSSFI |
| | QNTKSPLFMG | QNTKSPLFMG | DKGQELCADY |
| | KVVNPTQK | KVVNPTQK | SENTFTEYKK |
| | | | KLAERLKAKL |
| | | | PDATPKELAK |
| | | | LVNKRSDFAS |
| | | | NCCSINSPPL |
| | | | YCDSEIDAEL KNIL |

1-16. (canceled)

17. A method for diagnosing multiple sclerosis in a subject, the method, comprising determining the level of phosphorylation of a marker in a biological sample from the subject, wherein the marker is selected from α1-antitrypsin (a1AT) and vitamin D binding protein (VDBP); and comparing the level of phosphorylation of the marker in the sample to a reference value.

18. A method of claim 17, said method being carried out in vitro.

19. A method of claim 17, wherein the biological sample is a body fluid.

20. A method of claim 17, wherein the biological sample is a body fluid selected from blood, serum, plasma, cerebrospinal fluid, urine, and saliva.

21. A method of claim 17, wherein the marker is a1AT.

22. A method of claim 17, wherein the marker is VDBP.

23. A method of claim 17, wherein the marker is a1AT and VDBP.

24. A method of claim 17, wherein the reference value is the phosphorylation level of the marker in at least one sample from a non-multiple sclerosis subject.

25. A method of claim 17, wherein the level of phosphorylation of the marker is determined by detecting the presence of a metabolite.

26. A method of claim 17, wherein the subject is a lab animal.

27. A method of claim 17, wherein the subject is a human subject.

28. A method for monitoring the progression of multiple sclerosis in a subject, the method comprising measuring the level of phosphorylation of a marker in a biological sample from the subject in a first sample, wherein the marker is selected from the group consisting of a1AT and VDBP; measuring the level of phosphorylation of the marker in a biological sample from a second sample; and comparing the phosphorylation level of the marker measured in the first sample with the phosphorylation level of the marker measured in the second sample.

29. A method of assessing the efficacy of a treatment for multiple sclerosis in a subject, the method comprising comparing: (i) the phosphorylation level of a marker measured in a first sample obtained from the subject before the treatment has been administered to the subject, wherein the marker is selected a1AT and VDBP; and (ii) the phosphorylation level of the marker in a second sample obtained from the subject after the treatment has been administered to the subject, wherein a decrease in the phosphorylation level of the marker in the second sample relative to the first sample is an indication that the treatment is efficacious for treating multiple sclerosis in the subject.

30. A method for aiding in the diagnosis of multiple sclerosis in a subject, the method comprising determining the phosphorylation level of a marker in a biological sample from the subject, wherein the marker is selected from the group consisting of a1AT and VDBP; comparing the phosphorylation level of the marker in the sample to a reference value; and determining from the results of the comparison whether the subject is more or less likely to have multiple sclerosis.

31. A method for determining the type, stage or severity of multiple sclerosis in a subject, the method comprising determining the phosphorylation level of a marker in a biological sample from the subject, wherein the marker is selected from the group consisting of a1AT and VDBP; comparing the phosphorylation level of the marker in the sample to a reference value; and determining from the results of the comparison the type, stage or severity of multiple sclerosis in the subject.

32. A method for determining the risk of developing multiple sclerosis in a subject, the method comprising determining the phosphorylation level of a marker in a biological sample from the subject, wherein the marker is selected from a1AT and VDBP; comparing the phosphorylation level of the marker in the sample to a reference value; and determining from the results of the comparison that the subject has an increased or decreased risk of developing multiple sclerosis.
